(19) United States
(12) Patent Application Publication
(10) Pub. No.: US 2007/0082953 A1
(43) Pub. Date: Apr. 12, 2007

(54) Use of l-dopa, derivatives thereof and medicaments comprising said compounds for the prophylaxis of psychotic diseases

(76) Inventor: Rudolf-Giesbert Alken, Zepernick (DE)

Correspondence Address: KRIEGSMAN & KRIEGSMAN, 30 TURNPIKE ROAD, SUITE 9, SOUTHBOROUGH, MA, 01772, US

(21) Appl. No.: 10/539,793

(22) PCT Filed: Dec. 18, 2003
(86) PCT No.: PCT/DE03/04204

§ 371 Date: Nov. 18, 2006

(51) Int. Cl. A61K 31/198; A61K 31/137

(52) U.S. Cl. 514/567; 514/649

(57) ABSTRACT

The present invention concerns the use of L-dopa, its derivatives or physiologically compatible salts thereof for the prophylaxis of psychotic disorders and for the treatment of diseases which are caused by disrupted tyrosine transport or disrupted tyrosine decarboxylase. In addition, the invention concerns pharmaceutical compositions which contain L-dopa, its derivatives or physiologically compatible salts thereof for the prophylaxis of psychotic disorders, in addition to pharmaceutically compatible adjuvants and additives. In addition, the invention concerns the combination of L-dopa, its derivatives or physiologically compatible salts thereof with enzyme inhibitors for the prophylaxis of psychotic disorders and for the treatment of disorders which are caused by disrupted tyrosine transport or disrupted tyrosine decarboxylase, as well as corresponding pharmaceutical compositions for the prophylaxis of psychotic disorders and for the treatment of diseases which are caused by disrupted tyrosine transport or disrupted tyrosine decarboxylase.

The invention concerns the use of 3,4-dihydroxy-L-phenylalanine (L-dopa) and its derivatives for the production of pharmaceuticals as well as their use for the prophylaxis of psychotic disorders as well as for the treatment of diseases which are caused by disrupted tyrosine transport or disrupted tyrosine decarboxylase.

The symptoms of schizophrenic disorders are usually treated at the present time by neuroleptics such as chlorpromazine, haloperidol, sulpiride and their chemical relatives. Since treatment with neuroleptics does not cure the underlying disorder, a lapse in the treatment usually leads to a relapse for the patient.

Various psychic disorders are associated with a disturbance in the metabolism of noradrenaline, dopamine and serotonin.

L-dopa and its derivatives, particularly esters, have been utilized up to now, among other things, for the therapy of Parkinson's disease and of restless leg syndrome. L-dopa acts on the dopamine concentration in neurons of the brain. Unlike dopamine itself, it can pass through the blood-brain barrier and is converted to dopamine in the brain.

L-dopa is administered usually in pharmaceuticals with active additives. Combinations of L-dopa with peripheral decarboxylase inhibitors, with catechol-O-methyltransferase (COMT) inhibitors, with monoamine oxidase (MAO) inhibitors and with dopamine-β-hydroxylase inhibitors particularly find use.

In this connection, the decarboxylase inhibitors used are, for example: D,L-serine 2-(2,3,4-trihydroxybenzyl)hydrazide (benserazide), (−)-L-α-hydrazino-3,4-dihydroxy-α-methylhydrocinnamic acid (carbidopa), L-serine 2-(2,3,4-trihydroxybenzyl)hydrazide, glycine 2-(2,3,4-trihydroxybenzyl)hydrazide and L-tyrosine 2-(2,3,4-trihydroxybenzyl)hydrazide. Examples of combination preparations of L-dopa and decarboxylase inhibitors include, among others: Madopar® (L-dopa and benserazide hydrochloride) as well as Nacom® (L-dopa and carbidopa).

Examples of COMT inhibitors are entacapone (Comtan®) and cabergoline and frequently used MAO inhibitors are selegiline hydrochloride, moclobemide and tranylcypromine.

Calcium 5-butyl picolinate and calcium 5-pentyl picolinate are described as dopamine-β-hydroxylase inhibitors (DE-A-2,049,115).

According to the invention, use [of L-dopa] is preferred for relapse prophylaxis in psychotic disorders, particularly in schizophrenic disorders.

It was surprisingly found that L-dopa can be used for the prophylaxis of psychotic disorders. This is even more surprising since psychotic disorders are known to occur as side effects with high dosage of L-dopa.

The use of L-dopa, its derivatives and its physiologically compatible salts is preferred in combination with one enzyme inhibitor or several enzyme inhibitors for the prophylaxis of psychotic disorders as well as for the treatment of diseases which are caused by disrupted tyrosine transport or disrupted tyrosine decarboxylase.

It is advantageous if the enzyme inhibitor or the enzyme inhibitors involve decarboxylase inhibitors and/or catechol-O-methyltransferase inhibitors and/or monoamine oxidase inhibitors and/or β-hydroxylase inhibitors.

It is particularly advantageous if the decarboxylase inhibitor is selected from the group consisting of the following: D,L-serine 2-(2,3,4-trihydroxybenzyl)hydrazide (benserazide), (−)-L-α-hydrazino-3,4-dihydroxy-α-methylhydrocinnamic acid (carbidopa), L-serine 2-(2,3,4-trihydroxybenzyl)hydrazide, glycine 2-(2,3,4-trihydroxybenzyl)hydrazide and L-tyrosine 2-(2,3,4-trihydroxybenzyl)hydrazide as well as their physiologically compatible salts.

In particular, it is also advantageous if the catechol-O-methyltransferase inhibitor is selected from entacapone and cabergoline as well as physiologically compatible salts thereof.

It is also preferred if the monoamine oxidase inhibitor is selected from the group consisting of selegiline, moclobemide and tranylcypromine as well as physiologically compatible salts thereof.

In addition, it is particularly preferred if the β-hydroxylase inhibitor is selected from calcium 5-butyl picolinate and calcium 5-pentyl picolinate as well as their physiologically compatible salts.

Another subject of the invention is the use of L-dopa, its derivatives and physiologically compatible salts thereof for the production of pharmaceuticals for the prophylaxis of psychotic disorders as well as for the treatment of diseases which are caused by disrupted tyrosine transport or disrupted tyrosine decarboxylase.

Another subject of the present invention is a pharmaceutical composition, which contains L-dopa, its derivatives as well as physiologically compatible salts thereof for the prophylaxis of psychotic disorders as well as for the treatment of disorders which are caused by disrupted tyrosine transport or disrupted tyrosine decarboxylase, in addition to pharmaceutically compatible adjuvants and additives.

Particularly advantageous is a pharmaceutical composition which contains L-dopa, its derivatives as well as physiologically compatible salts thereof for the prophylaxis of psychotic disorders as well as for the treatment of diseases which are caused by disrupted tyrosine transport or disrupted tyrosine decarboxylase and one or more enzyme inhibitors, in addition to pharmaceutically compatible adjuvants and additives.

A pharmaceutical composition is particularly preferred in which the enzyme inhibitor or the enzyme inhibitors involve decarboxylase inhibitors and/or catechol-O-methyltransferase inhibitors and/or monoamine oxidase inhibitors and/or β-hydroxylase inhibitors.

Additionally preferred is a pharmaceutical composition in which the decarboxylase inhibitor is selected from the group consisting of D,L-serine 2-(2,3,4-trihydroxybenzyl)hydrazide (benserazide), (−)-L-α-hydrazino-3,4-dihydroxy-α-methylhydrocinnamic acid (carbidopa), L-serine 2-(2,3,4-trihydroxybenzyl)hydrazide, glycine 2-(2,3,4-trihydroxybenzyl)hydrazide and L-tyrosine 2-(2,3,4-trihydroxybenzyl)hydrazide as well as their physiologically compatible salts.

Particularly advantageous is a pharmaceutical composition in which the catechol-O-methyltransferase inhibitor is selected from entacapone and cabergoline as well as physiologically compatible salts thereof.

Additionally advantageous is a pharmaceutical composition in which the monoamine oxidase inhibitor is selected from the group consisting of selegiline, moclobemide and tranylcypromine as well as physiologically compatible salts thereof.

In addition, a pharmaceutical composition is preferred in which the β-hydroxylase inhibitor is selected from calcium 5-butyl picolinate and calcium 5-pentyl picolinate as well as physiologically compatible salts thereof.

The production of L-dopa and its derivatives such as, for example, the alkyl ester is known in and of itself.

For the production of physiologically compatible salts of L-dopa and its derivatives, the usual physiologically compatible inorganic and organic acids such as hydrochloric acid, hydrobromic acid, phosphoric acid, sulfuric acid, oxalic acid, maleic acid, fumaric acid, lactic acid, tartaric acid, malic acid, citric acid, salicylic acid, adipic acid and benzoic acid can be used. Additional acids that can be used are described, for example, in Fortschritte der Arzneimittelforschung, Vol. 10, pp. 224-225, Birkhäuser Publishers, Basel and Stuttgart (1966) and Journal of Pharmaceutical Sciences, Vol. 66, pp. 1-5 (1977).

The acid addition salts of L-dopa and its derivatives are usually obtained in a way known in and of itself by mixing the free base or solutions thereof with the corresponding acids or solutions thereof in an organic solvent, for example, a lower alcohol, such as methanol, ethanol, n-propanol or isopropanol or a lower ketone such as acetone, methyl ethyl ketone or methyl isobutyl ketone or an ether such as diethyl ether, tetrahydrofuran or dioxane. For better crystal precipitation, mixtures of the named solvents also can be used. In addition, physiologically compatible aqueous solutions of acid addition salts of the compounds used according to the invention can be produced therefrom in an aqueous acid solution.

The acid addition salts of L-dopa and its derivatives can be converted to the free base in a way known in and of itself, e.g., with alkalis or ion exchangers. Other salts can be obtained from the free base by reaction with inorganic or organic acids, particularly those which are suitable for the formation of therapeutically usable salts. These or also other salts of the new compound, such as, e.g., the picrate, may also serve for purification of the free base by converting the free base into a salt, separating this salt, and again releasing the base from the salt.

The subject of the present invention is also pharmaceuticals for oral, buccal, sublingual, nasal, rectal, subcutaneous, intravenous or intramuscular application as well as for inhalation, which, in addition to the usual vehicle and dilution agents, also contain L-dopa, a derivative, or the acid addition salt thereof as the active ingredient.

The pharmaceuticals of the invention are produced, in the known way and with suitable dosage, with the usual solid or liquid vehicle substances or dilution agents and the usually used pharmaceutical-technical adjuvants corresponding to the desired type of application. The preferred preparations consist of a form of administration which is suitable for oral application. Such forms of administration are, for example, tablets, sucking tablets, film tablets, dragees, capsules, pills, powders, solutions, aerosols or suspensions or slow-release forms.

Of course, parenteral preparations such as injection solutions are also considered. In addition, suppositories, for example, have also been named as preparations.

Corresponding tablets can be obtained, for example, by mixing the active substance with known adjuvants, for example, inert dilution agents such as dextrose, sugar, sorbitol, mannitol, polyvinylpyrrolidone, bursting agents such as corn starch or alginic acid, binders such as starches or gelantins, lubricants such as magnesium stearate or talc and/or agents for achieving a slow-release effect such as carboxypolymethylene, carboxymethylcellulose, cellulose acetate phthalate or polyvinyl acetate. The tablets may also consist of several layers.

In a corresponding manner, for the preparation of controlled or slow-release forms, dragees can also be produced by the coating of cores that have been produced analogously to tablets, by using agents commonly used in dragee coatings, for example, polyvinylpyrrolidone or shellac, gum arabic, talc, titanium dioxide or sugar. The dragee envelope may also consist of several layers, wherein the adjuvants mentioned above in the case of tablets can be used.

Solutions or suspensions containing the active substance used according to the invention may additionally contain agents that improve taste, such as saccharin, cyclamate or sugar, as well as, e.g., taste enhancers such as vanilla or orange extract. They may also contain suspension adjuvants such as sodium carboxymethylcellulose or preservatives such as p-hydroxybenzoate. Capsules containing active substances can be produced, for example, by mixing the active substance with an inert vehicle such as lactose or sorbitol and encapsulating this mixture in gelatin capsules.

Suitable suppositories can be produced, for example, by mixing with vehicle agents provided therefor, such as neutral fats or polyethylene glycol or derivatives thereof.

The production of the pharmaceutical preparations according to the invention is known in and of itself, and is described in handbooks known to the person skilled in the art, for example, Hager's Handbuch (5^{th} ed.) 2, 622-1045; List et al., Arzneiformenlehre [Instructions for Drug Forms], Stuttgart: Wiss. Verlagsges. 1985; Sucker et al., Pharmazeutische Technologie [Pharmaceutical Technology], Stuttgart; Thieme 1991; Ullmann's Enzyklopädie [Encyclopedia] (5th ed.) A 19, 241-271; Voigt, Pharmazeutische Technologie [Pharmaceutical Technology], Berlin: Ullstein Mosby 1995.

The following example explains the invention:

### EXAMPLE

Ten patients with acute schizophrenia, who had accordingly received maintenance therapy with fluphenazine for more than one year and were clinically stable, were successively taken off this drug. According to clinical experience (see Gitlin M., Nuechterlein K., Subotnik K. L. et al., Am. J. Psychiatry (2001) 158(11), pp. 1835-42), about 8 of the 10 patients would be expected to suffer a relapse within the following 12 months. Our patients were treated with L-dopa or a combination preparation containing L-dopa for 2 months prior to withdrawal and during the entire withdrawal phase. In these patients, a relapse occurred in only 2 of 10 cases.

1. Use of L-dopa, its derivatives and physiologically compatible salts thereof for the prophylaxis of psychoses, particularly also of schizophrenia psychoses, as well as for the treatment of diseases which are caused by disrupted tyrosine transport or disrupted tyrosine decarboxylase.

2. Use of L-dopa, its derivatives and physiologically compatible salts thereof for the production of pharmaceuticals for the prophylaxis of psychoses, particularly also of schizophrenia psychoses, as well as for the treatment of diseases which are caused by disrupted tyrosine transport or disrupted tyrosine decarboxylase.

3. Use of L-dopa, its derivatives and physiologically compatible salts thereof according to claim 1 or 2 in combination with at least one enzyme inhibitor.

4. Use of L-dopa, its derivatives and physiologically compatible salts thereof according to claim 3, further characterized in that the enzyme inhibitor(s) involve decarboxylase inhibitors and/or catechol-O-methyltransferase inhibitors and/or monoamine oxidase inhibitors and/or β-hydroxylase inhibitors.

5. Use of L-dopa, its derivatives and physiologically compatible salts thereof according to claim 4, further characterized in that the decarboxylase inhibitor is selected from the group consisting of D,L-serine 2-(2,3,4-trihydroxybenzyl)hydrazide (benserazide), (−)-L-α-hydrazino-3,4-dihydroxy-α-methylhydrocinnamic acid (carbidopa), L-serine 2-(2,3,4-trihydroxybenzyl)hydrazide, glycine 2-(2,3,4-trihydroxybenzyl)hydrazide and L-tyrosine 2-(2,3,4-trihydroxybenzyl)hydrazide as well as their physiologically compatible salts.

6. Use of L-dopa, its derivatives and physiologically compatible salts thereof according to claim 4, further characterized in that the catechol-O-methyltransferase inhibitor is selected from entacapone and cabergoline as well as physiologically compatible salts thereof.

7. Use of L-dopa, its derivatives and physiologically compatible salts thereof according to claim 4, further characterized in that the monoamine oxidase inhibitor is selected from the group consisting of selegiline, moclobemide and tranylcypromine as well as physiologically compatible salts thereof.

8. Use of L-dopa, its derivatives and physiologically compatible salts thereof according to claim 4, further characterized in that the β-hydroxylase inhibitor is selected from calcium 5-butyl picolinate and calcium 5-pentyl picolinate as well as physiologically compatible salts thereof.
